(19) United States
(12) Patent Application Publication
(10) Pub. No.: US 2021/0004593 A1
(43) Pub. Date: Jan. 7, 2021

(54) FOLIAR SAMPLING SYSTEM AND METHOD OF USE

(71) Applicant: Michelle Fisher, San Francisco, CA (US)

(72) Inventor: Michelle Fisher, San Francisco, CA (US)

(21) Appl. No.: 16/847,312

(22) Filed: Apr. 13, 2020

Related U.S. Application Data
(60) Provisional application No. 62/832,450, filed on Apr. 11, 2019.

(51) Int. Cl. G06K 9/00; G01N 33/00; G01J 3/28; A01B 79/00; A01B 79/02; B64C 39/02

(52) U.S. Cl. CPC G06K 9/00657; G01N 33/0098; G01J 3/2823; B64C 2201/127; A01B 79/02; B64C 39/024; A01B 79/005

(57) ABSTRACT

A foliage analysis system allows a farmer or another agricultural worker to obtain real-time or near real-time measurements of the health of plants or crops to take precise action to care for them. A machine with a spectrometer is used to evaluate a sample taken from a plant or field and then provide spectroscopy to a computer algorithm to determine the condition of the sample. The system also takes aerial spectral imagery to be used in conjunction with onsite handheld spectral measurements to determine the nutrients in the plants or the diseases afflicting them.

### BACKGROUND

### 1. Field of the Invention

The present invention relates generally to foliar sampling systems and methods, and more specifically, to a system with unique hardware, software, and other components for efficient foliar sampling to optimize crop fertilization and early identification of disease in a crop.

### 2. Description of Related Art

Foliar sampling systems are well known in the art and are effective means to determine various qualities of foliage, such as nutrient levels, health indexes, moisture levels, virus presence, microbial presence, and pests. Commonly, this information is obtained through wet chemical analysis in agricultural laboratories. The information obtained by these systems is used to guide fertilization applications, disease and pest mitigation and to enhance crop health. For example, FIG. 1 depicts a flowchart 101 of a conventional process. During use, the crop personnel will collect leaf samples from multiple trees or vines, package them into a container, such as a brown paper bag, and label the container, as shown with box 103. The samples are then shipped off to an agricultural lab for testing of the samples, which will be completed, and the results sent back, which can take weeks, as shown with boxes 105, 107. Due to the long turnaround time for agricultural lab test results, crop personal guess or estimate which fertilizers to apply, and in what concentrations, before test results are received. This guessing routinely results in decreased yield, crop damage due to unmitigated pest and disease pressures, and environmental damage from the application of excess fertilizers. The crop personnel will apply fertilizer before and after the results, which may not be suitable for the particular state of the crops, as shown with box 109.

One of the problems commonly associated with system 101 is its inefficiency. For example, the crops may change significantly between the time of the collection of a sample and the return of the results, making determined fertilizer use inaccurate.

Another problem commonly associated with the system 101 is that it is cost-prohibitive for the early detection and identification of specific crop disease across an entire field simultaneously.

Yet another problem commonly associated with the system 101 is that laboratory foliar testing is destructive, and once tested, the test sample is disposed of.

Accordingly, although great strides have been made in the area of foliage analysis, many shortcomings remain.

### DESCRIPTION OF THE DRAWINGS

The novel features believed characteristic of the embodiments of the present application are set forth in the appended claims. However, the embodiments themselves, as well as a preferred mode of use, and further objectives and advantages thereof, will best be understood by reference to the following detailed description when read in conjunction with the accompanying drawings, wherein:

FIG. 1 is a flowchart of a common foliage analysis system;

FIG. 2 is a simplified schematic of a foliage analysis system in accordance with a preferred embodiment of the present application;

FIG. 3 is a flowchart of a method of use of the system of FIG. 2;

FIG. 4 is a simplified schematic of an alternative embodiment of the system of FIG. 2; and

FIG. 5 is a flowchart of a method of establishing the health of a crop.

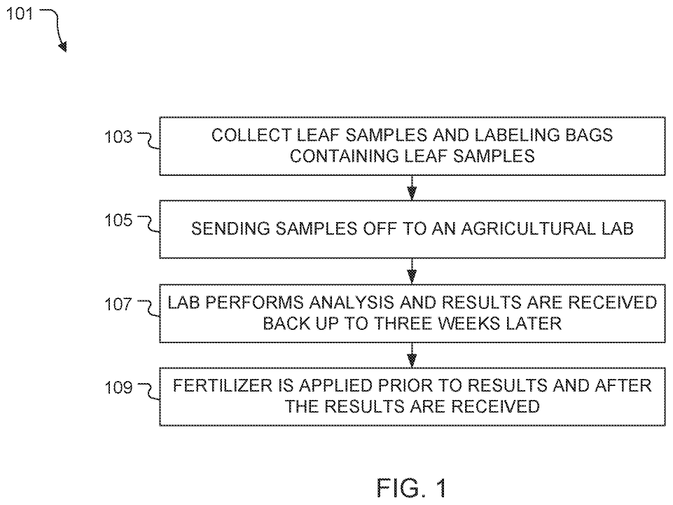

While the system and method of use of the present application is susceptible to various modifications and alternative forms, specific embodiments thereof have been shown by way of example in the drawings and are herein described in detail. It should be understood, however, that the description herein of specific embodiments is not intended to limit the invention to the particular embodiment disclosed, but on the contrary, the intention is to cover all modifications, equivalents, and alternatives falling within the spirit and scope of the present application as defined by the appended claims.

### DETAILED DESCRIPTION OF THE PREFERRED EMBODIMENT

Illustrative embodiments of the system and method of use of the present application are provided below. It will of course be appreciated that in the development of any actual embodiment, numerous implementation-specific decisions will be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The system and method of use in accordance with the present application overcomes one or more of the above-discussed problems commonly associated with conventional foliage analysis systems. Specifically, the present invention enables precise crop management through the quick and accurate means to collect and analyze data, and to thereby provide crop personnel with immediate test results in digital format. The invention of the present application digitizes nutrient and disease testing, and dramatically change the cost paradigm of the agricultural workers. The invention further allows each leaf sample to be measured non-invasively so and that the sample can be re-queried repeatedly as well as to allow other tests to be performed on that sample in perpetuity. These and other unique features of the system and method of use are discussed below and illustrated in the accompanying drawings.

The system and method of use will be understood, both as to its structure and operation, from the accompanying drawings, taken in conjunction with the accompanying description. Several embodiments of the system are presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless described otherwise.

The preferred embodiment herein described is not intended to be exhaustive or to limit the invention to the precise form disclosed. It is chosen and described to explain the principles of the invention and its application and practical use to enable others skilled in the art to follow its teachings.

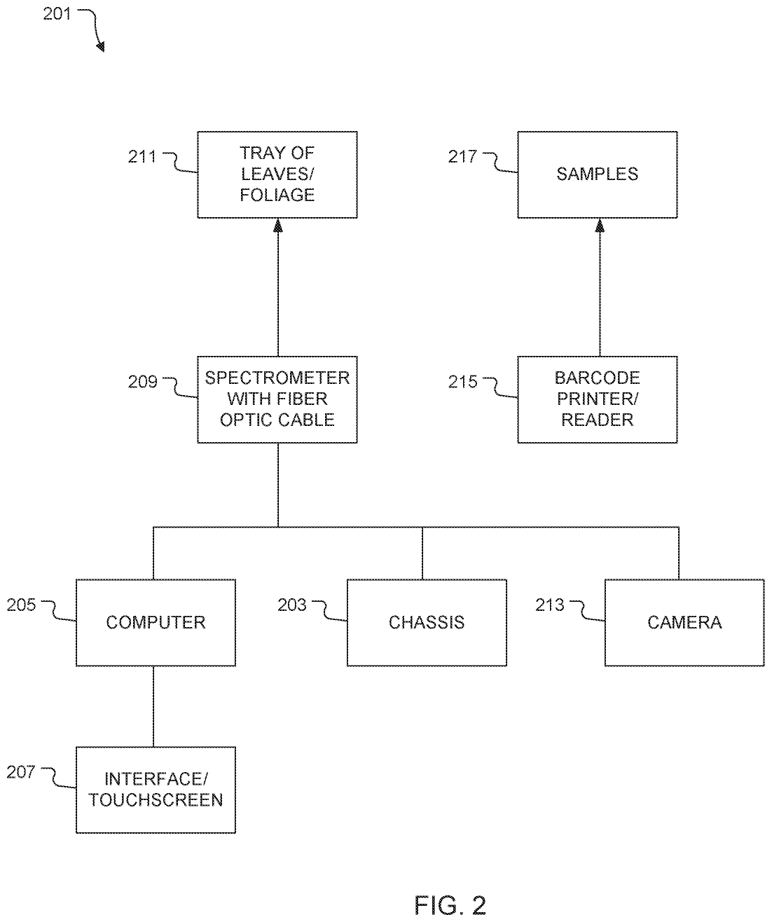

Referring now to the drawings wherein like reference characters identify corresponding or similar elements throughout the several views, FIG. 2 depicts a simplified schematic of a foliage analysis system 201 in accordance with a preferred embodiment of the present application. It will be appreciated that system 201 overcomes one or more of the above-listed problems commonly associated with conventional foliage analysis systems.

In the contemplated embodiment, system 101 includes a chassis 203 or other similar device that is configured to support and move a spectrometer 209 and work in conjunction with a computer 205 having an interface and/or touchscreen 207 for user manipulation. The computer 205 is configured to operate one or more algorithms and computer-readable mediums to collect and analyze data. The one or more algorithms of the present application are configured to digitally analyze, identify and quantify the micro and macronutrients such as nitrogen, potassium, phosphate or other substance present in the leaves. In one particular embodiment, the algorithms of the present application are also configured to produce a chlorophyll index. Other algorithms could be used in the detection and the identification of the highly-damaging RedBlotch, and LeafRoll grape viruses, as well as quantifying leaf moisture level and the like.

The chassis 203 is configured to receive commands from the user and operate the spectrometer with fiber optic cables 209 to collect data from a tray of leaves 211. It is contemplated that the chassis 203 could use step motors, worm gear or any other mechanism that controls the movement and position of the scanner. It is contemplated that the spectrometer 209 could also be a hyper-spectrometer or any other type of device that measures or records properties of the leaves/foliage through the reflectance of light waves.

In some embodiments, system 201 further includes one or more cameras 213 that are configured to collect additional data from the tray of leaves/foliage. The one or more cameras 213 can further work with the chassis 203 to guide targeting and aiming of the fiber optic cable of the spectrometer 209 to pre-programmed targets within the tray of leaves/foliage 211. Additionally, a lighting scheme could be used to improve the imagery or spectral measurements.

It is contemplated that lights, calibration devices or the like could be used to assist, augment or otherwise facilitate the operation of the spectrometer 209.

System 201 further includes one or more barcode printers/readers 215 that are configured to label and track samples 217 and can further be utilized to facilitate billing.

It should be appreciated that one of the unique features believed characteristic of the present application is the use of the combined features which function synergistically to provide clear, and near-instant actionable information derived from the digital analysis of the foliar leaf samples. This allows the user to quickly and efficiently determine the state of their crops to apply an accurate fertilizer treatment, which will increase crop yields while simultaneously screening for the presence of disease. The system further enables leaves to be tested to provide results in less than three minutes, which gives the grower information related to its nutrient levels or diseases present and to optimize fertilization or treatment plans to enhance crop vigor, maximize yield and profit. The ability to quickly know the status of the crop and thereby its fertilization needs without extended amounts of time between sample collection, test and receipt of test results. This gives crop personnel a new and needed level of control over reducing waste/costs, and the health and production of their crops.

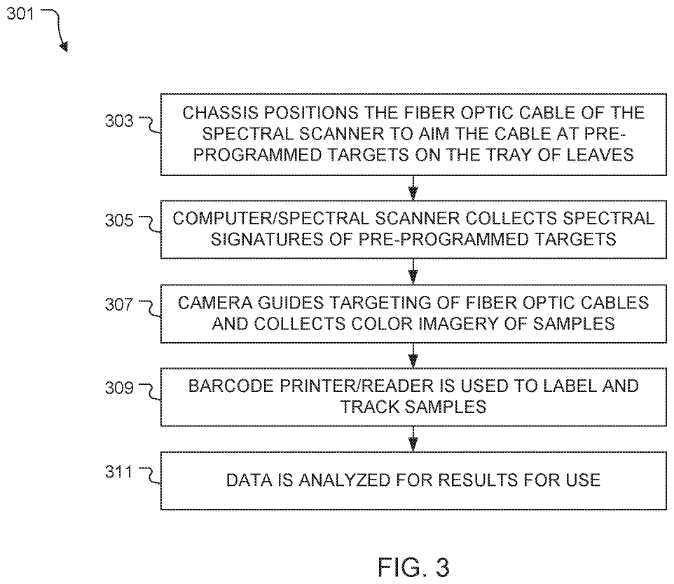

In FIG. 3, a flowchart 301 depicts a method of use associated with system 201. During use, the chassis is used to control the positioning of the fiber optic cable of the spectrometer to collect data from various pre-programmed targets over the tray of leaves, as shown with box 303. The computer collects the data from the spectrometer for analysis via one or more algorithms and/or computer-readable mediums, as shown with box 305. During this process, the one or more cameras (such as an SLR camera) are used to guide targeting of fiber optic cables and further to collect imagery of samples, as shown with box 307. It should be appreciated and understood that the combination multiple types of imagery can be used to aid in the determination of virus, bacterial, fungal and pest presence in the sample. The barcode printer/reader is used to label and track samples, and can further be used for billing purposes, as shown with box 309. The data is then processed and analyzed to create results that can easily be read and interpreted, as shown with box 311.

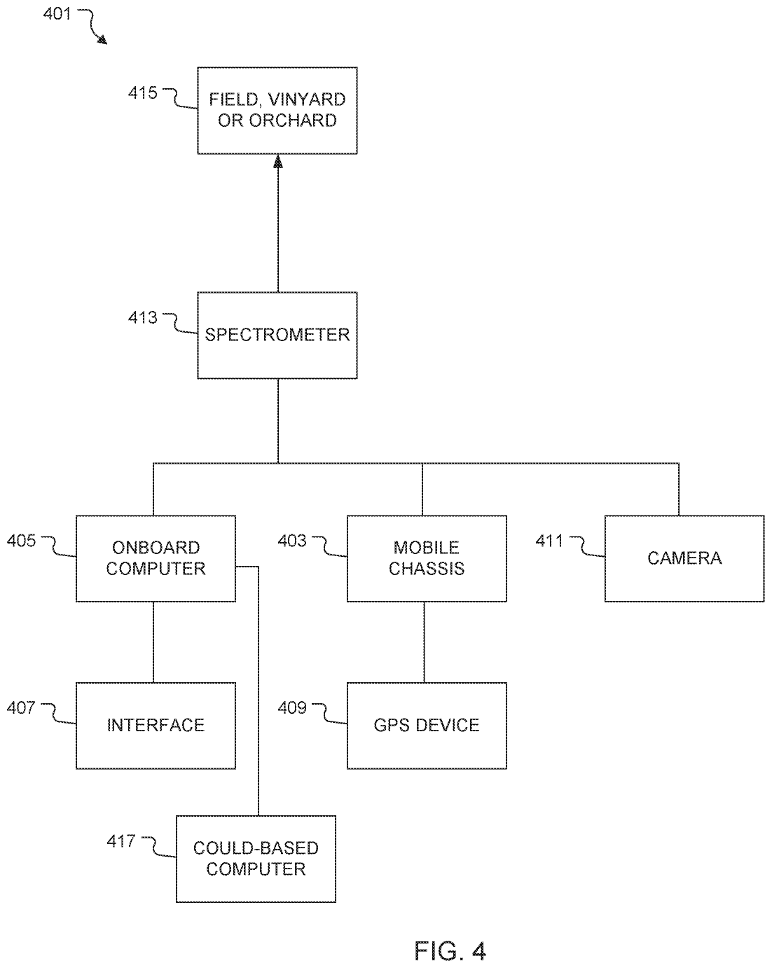

Referring now to FIG. 4 an alternative embodiment of the system 201 is depicted. Embodiment 401 includes a mobile chassis 403 that is contemplated to be an aerial platform that is equipped with a GPS device 409. The mobile chassis 403 supports or carries a spectrometer 413 and an onboard computer 405 having an interface 407 for user manipulation. It is further contemplated that the onboard computer 405 could be in digital communication with a cloud-based computer 417. The mobile chassis 403 includes a camera 411 to acquire images with the spectroscopy of a field, vineyard, orchard 415 or the like. The measurements taken by the spectrometer 413 are analyzed by the onboard or cloud-based computer and then provided to the user in a geospatial field map format or another readable medium.

It will be appreciated that the mobile chassis 403 with the spectrometer 413 and the mobile computer 405 enable instant onsite measurements of the plants thereabout. While an aerial platform is described it is contemplated that other types of chassis such as land-based or water-based could be used. Further, it is contemplated that features or tools could be included in the chassis such as shocks, gimbles, bumpers or the like to improve the function thereof.

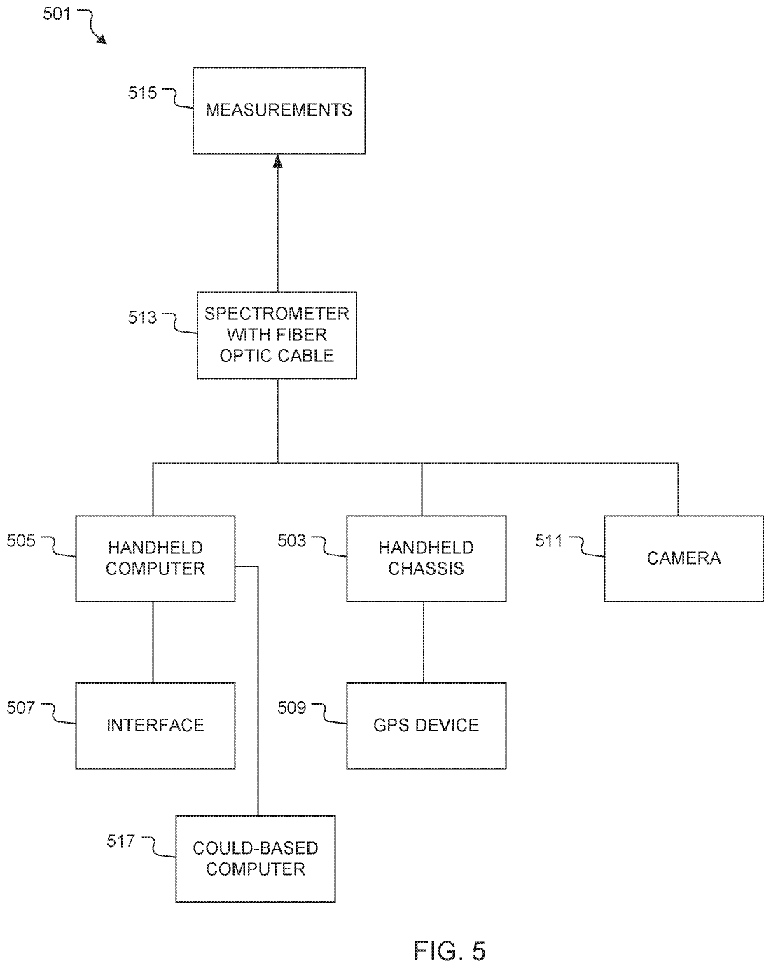

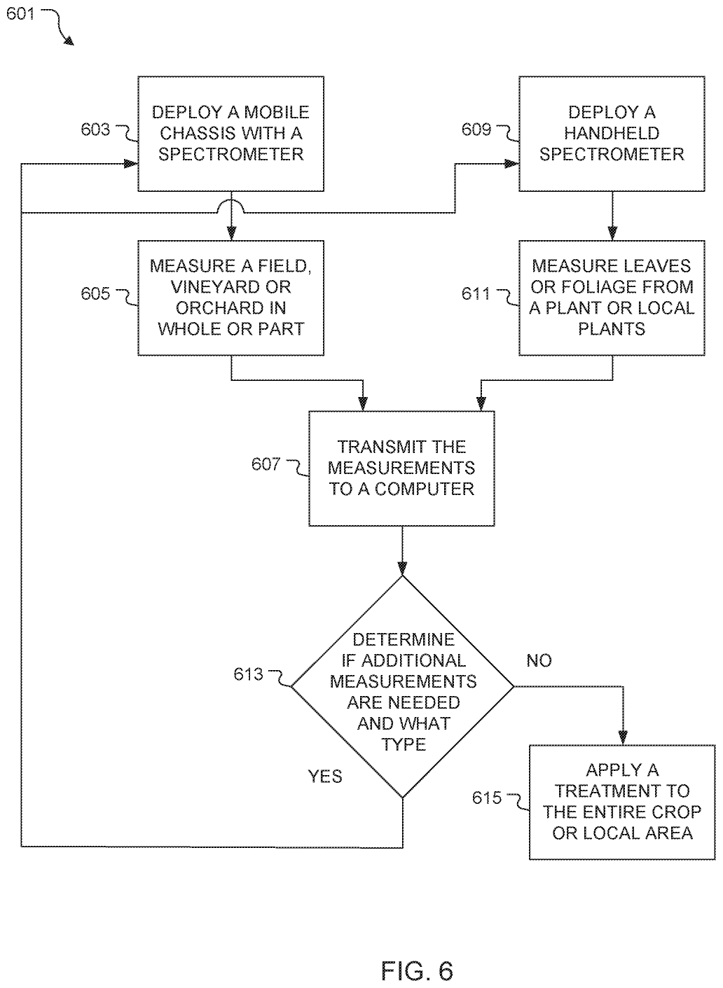

Referring now to FIG. 5 an alternative embodiment of the system 201 is depicted. Embodiment 501 includes a handheld chassis 503 that supports or carries a spectrometer with fiber optic cable 513 and is in digital communication with a handheld computer 505 having an interface 507 for user manipulation. It is further contemplated that the handheld computer 505 could be in digital communication with a cloud-based computer 517. The handheld computer 505 or handheld chassis 503 includes a camera 511 to acquire images with the spectroscopy of measurements 515 taken or the like. It will be appreciated that the handheld chassis 503 with the spectrometer 513 and the handheld computer 505 enable in-field measurements by crop personnel to nondestructively test the living, attached leaves of individual trees, vines or plants for the presence, and in some cases, identification of pre-symptomatic disease, generating results in near-real-time.

In a contemplated method of use, as depicted by FIG. 6 where system 401 and system 501 are used in conjunction to establish the health of a crop and enable precise management of the crop. The Method 601 includes deploying a mobile chassis with a spectrometer 603, measuring a field, vineyard or orchard in whole or part 605, transmitting the results to a handheld computer 607 or deploying a handheld spectrometer 609, measuring leaves or foliage from a plant or local plants 611, transmitting the measurements to a handheld computer 607, if as depicted by decision 613, additional measurements are needed determine the type and deploy the mobile chassis 603 or handheld scanner 609 as determined or if no additional measurements are needed applying a treatment to the entire crop or local area 615.

It will be apricated that the mobile chassis 403 and the handheld chassis 503 with their respective spectrometers provide a complete view of the whole crop and then allow the user to move to points of interest and take additional measurements as needed or desired. Additionally, in reverse, alocal measurement could be taken by the handheld chassis 503 and spectrometer 513 and then the mobile chassis 403 and spectrometer 413 are deployed to scan an area to determine the breadth of the local condition.

The particular embodiments disclosed above are illustrative only, as the embodiments may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. Although the present embodiments are shown above, they are not limited to just these embodiments, but are amenable to various changes and modifications without departing from the spirit thereof.

What is claimed:

1. A foliage analysis system comprising:
at least one spectrometer controlled by a chassis and a computer;
at least one tray of leaves/foliage;
at least one light source; and
at least one fiber optic cable;
wherein the at least one chassis and computer assist the spectrometer to measure the leaves/foliage in the at least one tray thereof via light waves; and
wherein the computer provides an analysis of the spectral measurements using one or more algorithms.

2. The system of claim 1 wherein the chassis and computer are mobile.

3. The system of claim 1 wherein the chassis and computer are handheld.

4. The system of claim 1 wherein the at least one camera provides imagery to compliment the spectral measurement.

5. The system of claim 1 wherein the computer exports spectral measurement data to a cloud-based computer for processing and generation of results reports.

6. A method of establishing the health of a crop, comprising:
deploying a mobile chassis with a spectrometer;
measuring a field, vineyard or orchard in whole or part; or
deploying a handheld spectrometer;
measuring leaves or foliage from a plant or local plants;
transmitting the measurements to a handheld computer;
if additional measurements are needed determining the type and deploy the mobile chassis or handheld scanner; or
if no additional measurements are needed applying a treatment to the entire crop or local area.
